(19) United States
(12) Patent Application Publication
(10) Pub. No.: US 2025/0366921 A1
(43) Pub. Date: Dec. 4, 2025

(54) TREATING INTRAVASCULAR OCCLUSIONS

(71) Applicant: Assaf PREISS, Shimshit (IL)

(72) Inventor: Assaf PREISS, Shimshit (IL)

(21) Appl. No.: 19/302,375

(22) Filed: Aug. 18, 2025

Related U.S. Application Data
(63) Continuation of application No. PCT/IB2024/051718, filed on Feb. 22, 2024.
(60) Provisional application No. 63/447,357, filed on Feb. 22, 2023.

(51) Int. Cl. A61B 18/26; A61B 17/00; A61B 18/00; A61B 18/22

(52) U.S. Cl. CPC A61B 18/26; A61B 2017/00061; A61B 2018/00244; A61B 2018/0041; A61B 2018/2211; A61B 2018/263

(57) ABSTRACT

Apparatus and methods are described for treating an occlusion in a blood vessel. A catheter is inserted into the blood vessel. An irradiation unit is driven to emit probing radiation such that the probing radiation is directed toward the occlusion, and returning radiation which is returned in response to the probing radiation impacting the occlusion, is detected. A composition of at least a portion of the occlusion is derived based on a signature that is indicative of the composition within the returning radiation. The irradiation unit is driven to irradiate the portion of the occlusion by emitting treatment radiation having a set of irradiation parameters. The set of irradiation parameters is determined to modulate between a photoacoustic effect of the treatment radiation and a photothermal effect of the treatment radiation, based on the composition of the portion of the occlusion. Other applications are also described.

### CROSS-REFERENCE TO RELATED APPLICATIONS

The present application is a continuation of PCT Application PCT/IB2024/051718 to Preiss (published as WO 24/176163), filed Feb. 22, 2024, entitled “Treating intravascular occlusions,” which claims priority from U.S. Provisional Application 63/447,357, entitled “System for detecting and treating arterial occlusions,” filed Feb. 22, 2023. The aforementioned applications are incorporated herein by reference.

### FIELD OF EMBODIMENTS OF THE INVENTION

Embodiments of the present invention are related generally to the field of medical devices and procedures, and specifically to the treatment of intravascular occlusions.

### BACKGROUND

Cardiovascular disease is a leading cause of mortality and a major contributor to disability. For example, an occlusion of an artery, such as a coronary artery, can be dangerous and potentially life-threatening. An occlusion can be detected via angiography (e.g., using fluoroscopy or intravascular ultrasound), and can be treated, for example, in an angioplasty procedure, which is often conducted under fluoroscopy. Chronic total occlusions are found in up to 20% of coronary angiographies and in more than 50% of peripheral angiographies. Heavily calcified arteries are found in around 30% of all angiographies.

Endovascular treatment, including angioplasty and atherectomy treatment, is currently the preferred modality for the treatment of both cardiac and peripheral arterial occlusive disease. Some atherectomy devices mechanically drill through the occlusion, while others use ultrasound waves to fragment the occlusion.

However, when an artery is severely narrowed and/or heavily calcified, and especially when the artery is totally occluded, the success rate of endovascular treatment declines dramatically, and the treatment is associated with a higher complication rate and prolonged procedure time. Failure to revascularize a vessel risks maintaining a state of debilitating angina or claudication, and often results in heart failure or limb amputation.

### SUMMARY

Embodiments of the present invention include a system for treating an occlusion in a blood vessel. The system includes a catheter, which is configured for insertion into the blood vessel, and one or more optical fibers, which pass through the catheter. The system further includes an irradiation unit configured to emit optical radiation such that the optical radiation is directed, by at least one of the optical fibers, toward the occlusion. The optical radiation can include probing radiation for probing the occlusion, e.g., so as to determine the composition of the occlusion, and/or treatment radiation for treating the occlusion, typically via a photothermal and/or photoacoustic effect.

In some embodiments, the system further includes an expandable element, which is coupled to the catheter or to one or more of the optical fibers and is configured to expand within the blood vessel, thereby defining a work area that includes a space between the expandable element and the occlusion. The system further includes at least one pump, which is configured to pump blood from the work area, via the catheter, while a liquid (e.g., saline) flows into the work area via the catheter. Subsequently to the pump beginning to pump the blood, the irradiation unit emits the optical radiation such that the optical radiation is directed, by the at least one of the optical fibers, through the work area and toward the occlusion.

In some embodiments, the expandable element includes an inflatable element, and the optical radiation is directed through the inflatable element. Alternatively or additionally, the distal end of the catheter is shaped to define a chamber, and the optical radiation is directed through the chamber.

In some embodiments, the composition of the occlusion is ascertained, e.g., using the aforementioned probing radiation. Based on multiple predefined sets of one or more irradiation parameters, a set of one or more irradiation parameters that corresponds to the composition is determined. Subsequently, the occlusion is irradiated, via one or more of the optical fibers, in accordance with the determined set. The predefined sets correspond to different respective materials and vary from each other with respect to a strength of the photoacoustic effect, relative to a strength of the photothermal effect, that the irradiation parameters provide. For example, the strength of the photoacoustic effect may vary across the predefined sets such that any first predefined set, which corresponds to a harder material, provides a stronger photoacoustic effect, relative to any second predefined set corresponding to a softer material.

There is therefore provided, in accordance with some embodiments of the present invention, a system for treating an occlusion in a blood vessel. The system includes a catheter, configured for insertion into the blood vessel, one or more optical fibers, which pass through the catheter, an expandable element coupled to the catheter or to one or more of the optical fibers and configured to expand within the blood vessel, thereby defining a work area that includes a space between the expandable element and the occlusion, at least one pump, configured to pump blood from the work area, via the catheter, while a liquid flows into the work area via the catheter, and an irradiation unit, configured to emit optical radiation, subsequently to the pump beginning to pump the blood, such that the optical radiation is directed, by at least one of the optical fibers, through the work area and toward the occlusion.

In some embodiments, the pump is further configured to pump the liquid into the work area.

In some embodiments, the at least one of the optical fibers is configured to direct the optical radiation at least partly laterally toward the occlusion.

In some embodiments, the expandable element is configured to expand upstream from the occlusion such that the expandable element, once expanded, inhibits a flow of blood into the work area.

In some embodiments, the system further includes a camera configured to image the occlusion through the work area subsequently to the pump beginning to pump the blood.

In some embodiments, the optical radiation includes treatment radiation for treating the occlusion.

In some embodiments, the at least one of the optical fibers is configured to advance from the catheter prior to directing the optical radiation.

In some embodiments,
the expandable element is a first expandable element and the space is a first space,
the first expandable element is configured to expand at a first side of the occlusion,
the system further includes a second expandable element configured to expand at a second side of the occlusion, such that the work area further includes a second space between the second expandable element and the occlusion, and
the irradiation unit is configured to emit at least some of the optical radiation while the first expandable element and the second expandable element are expanded.

In some embodiments,
the catheter includes an outer tube and an inner tube that passes through, and is deployable from, the outer tube, and
the expandable element is coupled to the inner tube.

In some embodiments, the at least one of the optical fibers is configured to advance through the inner tube prior to directing the optical radiation.

In some embodiments, the optical radiation includes probing radiation for probing the occlusion.

In some embodiments, at least one of the optical fibers is configured to receive, through the work area, other radiation that reflects, scatters, or fluoresces from the occlusion in response to the probing radiation.

There is further provided, in accordance with some embodiments of the present invention, a method for treating an occlusion in a blood vessel. The method includes inserting a catheter, through which pass one or more optical fibers, into the blood vessel. The method further includes, subsequently to inserting the catheter, expanding an expandable element coupled to the catheter or to one or more of the optical fibers, thereby defining a work area that includes a space between the expandable element and the occlusion. The method further includes, subsequently to expanding the expandable element, pumping blood from the work area, via the catheter, while a liquid flows into the work area via the catheter. The method further includes, subsequently to beginning the pumping of the blood, directing optical radiation, via at least one of the optical fibers, through the work area and toward the occlusion.

In some embodiments, expanding the expandable element includes expanding the expandable element within 1 cm from the occlusion.

There is further provided, in accordance with some embodiments of the present invention, a system for treating an occlusion in a blood vessel. The system includes a catheter, configured for insertion into the blood vessel, one or more optical fibers, which pass through the catheter, an inflatable element coupled to the catheter or to one or more of the optical fibers, and configured to inflate within the blood vessel, and an irradiation unit, configured to emit optical radiation, subsequently to the inflation of the inflatable element, such that the optical radiation is directed, by at least one of the optical fibers, through the inflatable element and toward the occlusion.

In some embodiments,
the catheter includes an outer tube and an inner tube that passes through, and is deployable from, the outer tube, and
the inflatable element is coupled to the inner tube.

In some embodiments, the at least one of the optical fibers is configured to direct the optical radiation at least partly laterally through the inflatable element.

In some embodiments, the system further includes a camera configured to image the occlusion through the inflatable element.

In some embodiments, the optical radiation includes treatment radiation for treating the occlusion.

In some embodiments, the optical radiation includes probing radiation for probing the occlusion.

In some embodiments, at least one of the optical fibers is configured to receive, through the inflatable element, other radiation that reflects, scatters, or fluoresces from the occlusion in response to the probing radiation.

In some embodiments, the inflatable element is further configured to push the occlusion toward a wall of the blood vessel.

There is further provided, in accordance with some embodiments of the present invention, a method for treating an occlusion in a blood vessel. The method includes inserting a catheter, through which pass one or more optical fibers, into the blood vessel. The method further includes, subsequently to inserting the catheter, inflating an inflatable element coupled to the catheter or to one or more of the optical fibers. The method further includes, subsequently to inflating the inflatable element, directing optical radiation, via at least one of the optical fibers, through the inflatable element and toward the occlusion.

In some embodiments, directing the optical radiation through the inflatable element includes directing the optical radiation through the inflatable element while the inflatable element contacts the occlusion.

There is further provided, in accordance with some embodiments of the present invention, an apparatus for use with an irradiation unit configured to emit optical radiation. The apparatus includes a catheter configured for insertion into a blood vessel containing an occlusion, a distal end of the catheter being shaped to define a chamber, and one or more optical fibers passing from the irradiation unit, through the catheter, to the distal end of the catheter, and configured to direct the optical radiation through the chamber and toward the occlusion.

In some embodiments, the chamber contains a fluid.

In some embodiments, the fluid includes air.

In some embodiments, the fluid includes saline.

In some embodiments, the chamber is lateral to respective distal ends of the optical fibers.

In some embodiments, the apparatus further includes a camera configured image the occlusion through the chamber.

In some embodiments, the optical radiation includes treatment radiation for treating the occlusion.

In some embodiments, the optical radiation includes probing radiation for probing the occlusion.

In some embodiments, the optical fibers are further configured to receive other radiation, which reflects, scatters, or fluoresces from the occlusion in response to the probing radiation, through the chamber.

There is further provided, in accordance with some embodiments of the present invention, a method, including inserting a catheter into a blood vessel containing an occlusion, a distal end of the catheter being shaped to define a chamber, and, using an irradiation unit, emitting optical radiation through one or more optical fibers passing from the irradiation unit, through the catheter, to the distal end of the catheter, such that the optical fibers direct the optical radiation through the chamber and toward the occlusion.

There is further provided, in accordance with some embodiments of the present invention, a system for treating an occlusion in a blood vessel. The system is for use with an irradiation unit and includes a catheter, configured for insertion into the blood vessel, one or more optical fibers, which pass through the catheter, and one or more processors. The processors are configured to ascertain a composition of the occlusion, to determine, based on multiple predefined sets of one or more irradiation parameters, a set of one or more irradiation parameters that corresponds to the composition, and to irradiate the occlusion via the optical fibers, using the irradiation unit, in accordance with the determined set. The predefined sets correspond to different respective materials and vary from each other with respect to a strength of a photoacoustic effect, relative to a strength of a photothermal effect, that the irradiation parameters provide.

In some embodiments, the strength of the photoacoustic effect varies across the predefined sets such that any first predefined set, which corresponds to a harder material, provides a stronger photoacoustic effect, relative to any second predefined set corresponding to a softer material.

There is further provided, in accordance with some embodiments of the present invention, a method for treating an occlusion in a blood vessel. The method includes ascertaining a composition of the occlusion, determining, based on multiple predefined sets of one or more irradiation parameters, a set of one or more irradiation parameters that corresponds to the composition, and irradiating the occlusion, via one or more optical fibers, in accordance with the determined set. The predefined sets correspond to different respective materials and vary from each other with respect to a strength of a photoacoustic effect, relative to a strength of a photothermal effect, that the irradiation parameters provide.

There is further provided, in accordance with some embodiments of the present invention, an apparatus for use with an irradiation unit. The apparatus includes a catheter, configured for insertion into a blood vessel of a subject, and multiple optical fibers that pass through the catheter from a proximal end of the catheter to a distal end of the catheter and are configured to direct optical radiation from the irradiation unit toward an occlusion in the blood vessel, at least two of the optical fibers being fused to one another at the proximal end of the catheter and/or at the distal end of the catheter, but not between the proximal end and the distal end.

There is further provided, in accordance with some embodiments of the present invention, an apparatus for use with an irradiation unit. The apparatus includes at least one optical fiber configured to direct optical radiation from the irradiation unit toward an occlusion in a blood vessel, and an expandable element coupled to the optical fiber and configured to expand within the blood vessel prior to the directing of the optical radiation.

In some embodiments, the expandable element is configured to expand until the expandable element contacts an inner wall of the blood vessel over an entire circumference of the blood vessel.

In some embodiments, the expandable element is configured to uncouple from the optical fiber while expanding.

In some embodiments, the expandable element includes an inflatable element coupled to a distal end of the optical fiber and configured to inflate within the blood vessel such that, following the inflation of the inflatable element, the optical radiation is directed through the inflatable element.

The present invention will be more fully understood from the following detailed description of embodiments thereof, taken together with the drawings, in which:

### BRIEF DESCRIPTION OF THE DRAWINGS

FIG. 1 is a schematic illustration of a system for treating an occlusion in a blood vessel of a subject, in accordance with some embodiments of the present invention;

FIG. 2A and FIG. 2B schematically illustrate different types of optical fibers, in accordance with some embodiments of the present invention;

FIG. 3A and FIG. 3B schematically illustrate a frontal view of the distal end of a catheter, in accordance with some embodiments of the present invention;

FIG. 3C is a schematic illustration of a bundle of optical fibers, in accordance with some embodiments of the present invention;

FIG. 4 is a schematic illustration of a catheter with a coupled expandable element, in accordance with some embodiments of the present invention;

FIG. 5A, FIG. 5B, FIG. 5C, FIG. 5D, FIG. 5E, FIG. 5F, and FIG. 5G are schematic illustrations of uses of an expandable element, in accordance with some embodiments of the present invention;

FIG. 5H is a schematic illustration of a use of two expandable elements, in accordance with some embodiments of the present invention;

FIG. 6A is a schematic illustration of a frontal view of the distal end of a catheter, in accordance with some embodiments of the present invention;

FIG. 6B is a schematic illustration of a side view of the distal end of a catheter, in accordance with some embodiments of the present invention;

FIG. 7 is a schematic illustration of optical fibers connected to a console, in accordance with some embodiments of the present invention;

FIG. 8 shows a flow diagram for a method for treating an occlusion, in accordance with some embodiments of the present invention; and

FIG. 9A, FIG. 9B, FIG. 9C, and FIG. 9D show experimental results illustrating the varying responses of different materials to irradiation, thereby demonstrating the utility of embodiments of the present invention.

### DETAILED DESCRIPTION

### Overview

Conventional devices for treating intravascular occlusions suffer from several shortcomings. First, these devices often require passing a guidewire across the occlusion, but if the vascular lumen is totally occluded, this can be challenging. For example, although the guidewire can be passed through a false lumen or a retrograde lumen, these techniques are time-consuming, difficult to master, and risky. Second, conventional devices are designed for debulking chemically-homogenous occlusions, but in practice, an occlusion may include multiple different types of materials such as collagen, lipids, thrombus, and calcified plaque, which may require different treatment mechanisms. Moreover, prolonged fluoroscopy usage during angiography and/or treatment leads to radiation-related risks.

Hence, embodiments of the present invention provide an intravascular device configured to facilitate treating an intravascular occlusion without the above shortcomings. The device comprises a catheter and one or more optical fibers (comprising silica, for example), which pass through the catheter. The optical fibers are configured to direct optical radiation, which is emitted by one or more extracorporeal irradiation units, at the occlusion. In some embodiments, the irradiation units comprise a treatment irradiation unit configured to emit treatment radiation for ablating, coagulating, and/or fragmenting the occlusion. Alternatively or additionally, the irradiation units comprise a probing irradiation unit configured to generate probing radiation for probing the occlusion, e.g., so as to determine the composition of the occlusion and/or the distance of the occlusion from the optical fibers. For example, following a probing irradiation of the occlusion, radiation returning from the occlusion-including reflected, scattered, and/or fluoresced radiation—may be sensed by a camera, such as a charge-coupled device camera, or carried, by the optical fibers, outside the body for analysis.

For example, in some embodiments, after advancing the catheter through the blood vessel, the occlusion is probed. In response to the probing, parameters of the treatment radiation, such as the power and/or wavelength of each pulse of radiation, are determined so as to achieve a desired effect. For example, the desired effect for a softer occlusion, such as a blood clot, is typically thermal ablation or coagulation, whereas for a harder occlusion, such as plaque, the desired effect is typically fragmentation via a photoacoustic effect. Subsequently, the occlusion is treated with the parameters. Thus, an advantage of embodiments of the present invention is the real-time determination of the occlusion properties and the adaptation of the treatment procedure to these properties.

In some embodiments, the catheter comprises one or more working channels. In some such embodiments, a liquid, which typically includes a contrast dye and/or saline, is delivered (e.g., infused or pumped) through one of the working channels into the blood vessel, such that the liquid dilutes the subject's blood near the occlusion. (In the context of the present application, including the claims, the “dilution” of blood refers to the replacement of some or all of the blood with another liquid.) Advantageously, the liquid increases visibility of the occlusion, enhances the aforementioned photoacoustic effect, cools the tissue, and/or provides a more stable medium for the passage of optical radiation, relative to blood.

Alternatively or additionally, suction is applied, e.g., by a suction pump, via one of the working channels. The suction facilitates the dilution of the blood and/or helps remove fragments of the occlusion from the blood vessel. In some embodiments, the suctioned blood is filtered and returned to the subject's bloodstream, e.g., via infusion. In some embodiments, a single pump suctions the blood and also pumps the liquid.

In some embodiments, the device further comprises an expandable element, such as an inflatable element (e.g., a balloon) or a stent (e.g., a stent having an elastic cover), coupled to the catheter or to one or more of the optical fibers. The expandable element is expanded near the occlusion. In some embodiments, the expandable element stabilizes and, typically, centers the catheter or optical fiber(s) within the blood vessel. Alternatively or additionally, the expandable element impedes the flow of blood into the vicinity of the occlusion, thereby facilitating the dilution of the blood.

For some embodiments in which the expandable element comprises an inflatable element, the inflatable element is inflated with a fluid (e.g., air or saline) that provides a suitable medium for the transfer of optical radiation. Subsequently to the inflation of the inflatable element, probing and/or treatment radiation is directed at the occlusion via the inflatable element, and/or the occlusion is imaged via the inflatable element. Alternatively or additionally, the distal end of the catheter is shaped to define a vacuum chamber or a chamber filled with any suitable fluid, such as air or saline. The optical radiation is directed at the occlusion via the chamber, and/or the occlusion is imaged via the chamber. Advantageously, the inflatable element or the chamber increases visibility of the occlusion and/or provides a more stable medium for the passage of optical radiation, relative to blood.

### System Description

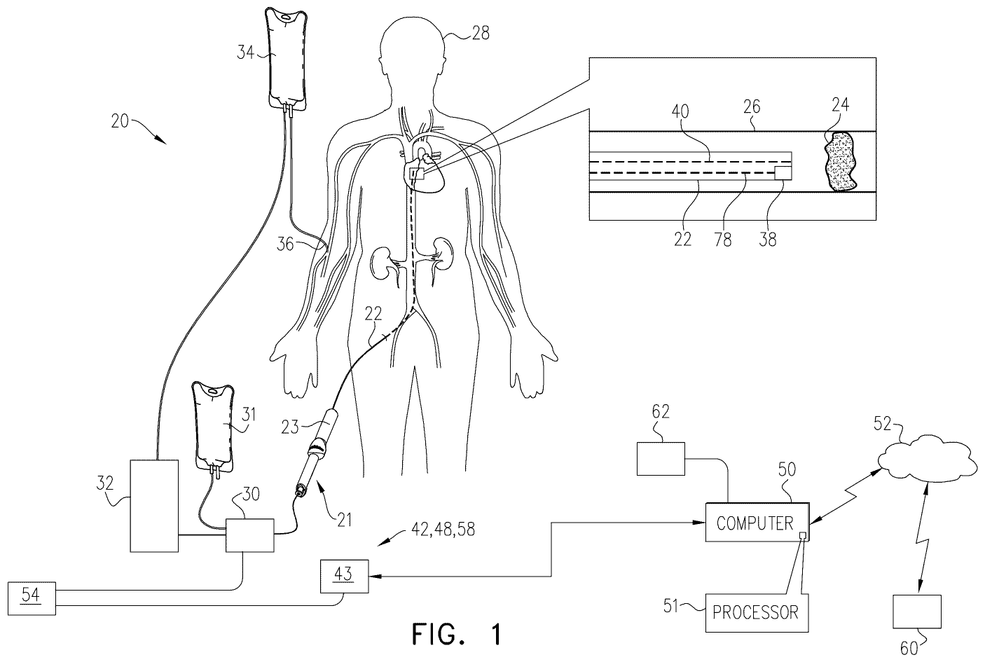

Reference is initially made to FIG. 1, which is a schematic illustration of a system 20 for treating an occlusion 24, such as plaque or a blood clot, in a blood vessel 26, such as a coronary artery or a peripheral artery, of a subject 28, in accordance with some embodiments of the present invention.

System 20 comprises at least one treatment apparatus 21 comprising a catheter 22 configured for insertion into blood vessel 26. In some embodiments, blood vessel 26 is accessed via a radial artery or femoral artery, each of which connects to the ascending aorta of subject 28. Typically, catheter 22 enters blood vessel 26 from the upstream direction, such that the catheter is advanced toward occlusion 24 in the direction of blood flow.

Typically, apparatus 21 is controlled, by the user, via a control handle 23 near the proximal end of catheter 22. In some embodiments, catheter 22 comprises one or more radiopaque markers, which facilitate positioning and/or orienting the catheter. Alternatively or additionally, the positioning and/or orienting is facilitated by one or more electromagnetic sensors, such as a coil surrounding the distal end of the catheter. In some embodiments, catheter 22 comprises one or more sensors for sensing temperature and/or pressure.

Typically, system 20 typically further comprises at least one pump 30. In some embodiments, pump 30 is configured to pump a liquid 31, such as saline and/or contrast dye, into the blood vessel via catheter 22. Alternatively or additionally, pump 30 is configured to pump blood (which may be mixed with the aforementioned liquid) from the blood vessel, via catheter 22. In some embodiments, a filter 32 filters any fragments of the occlusion from the suctioned blood, a blood collection bag 34 holds the filtered blood, and an intravenous tube 36 returns the filtered blood from blood collection bag 34 to the subject.

In some embodiments, apparatus 21 further comprises a camera 38, such as a charge-coupled device camera, configured to image occlusion 24. In some such embodiments, as assumed in FIG. 1, camera 38 is disposed within the catheter at the distal end of the catheter, and is connected to circuitry at the proximal end of the catheter via wiring 78. (For simplicity, wiring 78 is omitted from the subsequent drawings.)

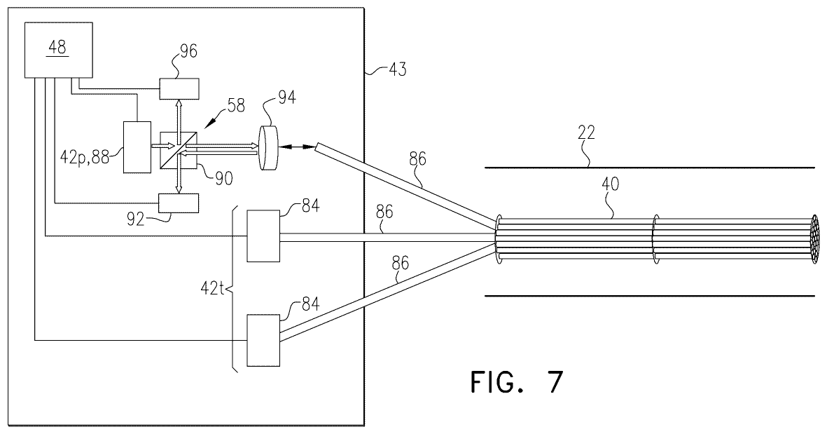

System 20 further comprises one or more irradiation units 42 configured to emit optical radiation. In some embodiments, irradiation units 42 belong to a console 43, which is shown in more detail in FIG. 7. Apparatus 21 further comprises one or more optical fibers 40, which pass from irradiation units 42, through the catheter, to the distal end of the catheter. Optical fibers 40 are configured to direct the emitted radiation toward occlusion 24.

Typically, irradiation units 42 comprise a probing irradiation unit 42p (FIG. 7), which is configured to emit probing radiation for probing the occlusion. In some embodiments, the probing radiation is directed at the occlusion by the optical fibers, and the returning radiation, including reflected, scattered, and/or fluoresced radiation, which is carried proximally by the optical fibers, is analyzed so as to ascertain the composition and/or distance of the occlusion. Alternatively or additionally, the probing radiation illuminates the field of view of camera 38, thereby facilitating the imaging of the occlusion.

In some embodiments, the probing radiation also facilitates navigation of the catheter. For example, the catheter may be navigated based on imaging performed by camera 38, which, as noted above, is facilitated by the probing radiation. Alternatively or additionally, the probing radiation is directed from the catheter, and based on the power of the returning radiation, distances to various objects in the vicinity of the catheter, such as blood-vessel walls, are ascertained.

In some embodiments, the probing irradiation unit comprises a low-power multi-wavelength pulsed or constant-wave laser array operating, for example, at wavelengths of 200-3000 nm, a pulse duration (or “width”) of 10 ns-1 s, an average power of 0.1 mW-5 W, and/or a peak power of up to 0.5 kW. Examples of suitable laser types include solid-state lasers (e.g., diode-pumped solid-state lasers), diode lasers, and fiber lasers. Alternatively or additionally, the probing irradiation unit comprises a broadband (white-radiation) spectrum source such as a xenon lamp, a halogen lamp, a tungsten lamp, a tungsten-halogen lamp, or a deuterium lamp, which emits radiation at wavelengths between 185 and 5000 nm, for example. Alternatively or additionally, the probing irradiation unit comprises one or more light emitting diodes (e.g., multi-wavelength light emitting diodes), configured to illuminate the field of view of the camera.

Alternatively or additionally to the probing irradiation unit, irradiation units 42 comprise a treatment irradiation unit 42t (FIG. 7), which is configured to emit treatment radiation for treating the occlusion. In some embodiments, the treatment irradiation unit comprises one or more lasers. For example, in some embodiments, the treatment irradiation unit comprises a high-power pulsed laser and a mid-power continuous-wave laser, which optionally operate at different wavelengths. In some embodiments, treatment irradiation unit 42t operates at wavelengths of 270-3150 nm, a pulse duration of 10 ns-1 s, an average power of up to 120 W, and/or a peak power of up to 20 kW. Examples of suitable laser types include solid-state lasers (e.g., diode-pumped solid-state lasers), diode lasers, and fiber lasers.

In some embodiments, the treatment irradiation unit is also configured to emit radiation at lower energies. In such embodiments, the lower-energy radiation may be used for probing, such that a separate probing irradiation unit may not be required.

In some embodiments, camera 38 is disposed within the catheter at the proximal end of the catheter, or proximally to the catheter. In such embodiments, optical fibers 40 carry optical radiation from the vicinity of the occlusion to the camera.

In some embodiments, at least some optical fibers 40 are moveable axially and/or radially with respect to the catheter. For example, in some embodiments, the distal ends of at least some optical fibers can be flexed using control handle 23. In some embodiments, the diameter of each optical fiber is between 50 and 2000 μm.

Typically, system 20 further comprises a processing unit (or “processor”) 48, which, in some embodiments, also belongs to console 43. Typically, processing unit 48 is configured to perform at least some of the processing functionality described herein cooperatively with a processor 51 belonging to a computer 50. Alternatively or additionally, processing unit 48 communicates output to processor 51. In response thereto, processor 51 logs, communicates, and/or displays the output on a display 62, which typically comprises a touch screen. For example, processor 51 may display an output suggesting how the treatment should proceed. Using display 62, the user can view any images, monitor the procedure, and/or confirm or set any relevant parameters. In some embodiments, processor 51 is configured to exchange communication, e.g., with a big-data analytics unit 60, over the Internet 52.

As further described below with reference to FIG. 7, in some embodiments, processing unit 48 is configured to analyze (typically, in real-time, e.g., within several milliseconds) any radiation returning from the occlusion via optical fibers 40. For example, based on the energy of the returning radiation, the processing unit may calculate the distance between the optical fibers and the occlusion. (A higher energy indicates a smaller distance.) Alternatively or additionally, based on the energy of the returning radiation at various wavelengths, the processing unit may distinguish between the occlusion and healthy tissue and/or determine the composition of the occlusion, thereby facilitating selecting the optimal parameters for treatment.

Alternatively or additionally, processing unit 48 controls the emission of the optical radiation from irradiation units 42, typically in real-time. Alternatively or additionally, the processing unit controls any safety mechanisms. For example, in some embodiments, the processing unit processes a signal from a temperature sensor at the distal end of the catheter. If the signal indicates that the blood vessel is beginning to overheat, the processing unit pauses the treatment (e.g., by controlling an electromechanical shutter) and/or generates an alert. Alternatively or additionally, the processing unit monitors the treatment for efficacy, and decides when the occlusion has been sufficiently treated. Alternatively or additionally, processing unit 48 controls camera 38, synchronizes the camera with irradiation units 42, and/or calibrates optical fibers 40. Alternatively or additionally, processing unit 48 assists the user perform the procedure. For example, the processing unit may guide the user in positioning the distal ends of optical fibers 40, suggest suitable laser parameters (e.g., wavelength, pulse width, and/or pulse energy), and/or provide feedback regarding the efficacy of the treatment.

In some embodiments, system 20 further comprises a hardware control unit 54 configured to control other hardware components of the system, such as pump 30 and/or irradiation units 42. For example, control unit 54 may control the cooling of the treatment irradiation unit. Alternatively or additionally, the control unit may control the voltage or current supplied to other components of the system. In some embodiments, control unit 54 is configured via computer 50.

System 20 further comprises optical components 58, which, in some embodiments, also belong to console 43. As further described below with reference to FIG. 7, optical components 58 typically comprise one or more spectrometers and/or one or more optical detectors (or “sensors”), which facilitate the analysis performed by processing unit 48. In some embodiments, the spectrometers and/or optical detectors vary in type, so as to be suitable for various different wavelengths. Examples of optical detector materials include silicon, indium antimony, and indium gallium arsenide. In some embodiments, optical components 58 further comprise one or more mirrors, beam combiners, beam splitters, and/or lenses.

In general, each of the processors described herein, including processing unit 48 and processor 51, may be embodied as a single processor or as a cooperatively networked or clustered set of processors. The functionality of the processor may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, the processor may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the processor in electronic form, over a network, for example. Alternatively or additionally, the program code and/or data may be provided and/or stored on non-transitory tangible media, such as magnetic, optical, or electronic memory. Such program code and/or data, when provided to the processor, produce a machine or special-purpose computer, configured to perform the tasks described herein.

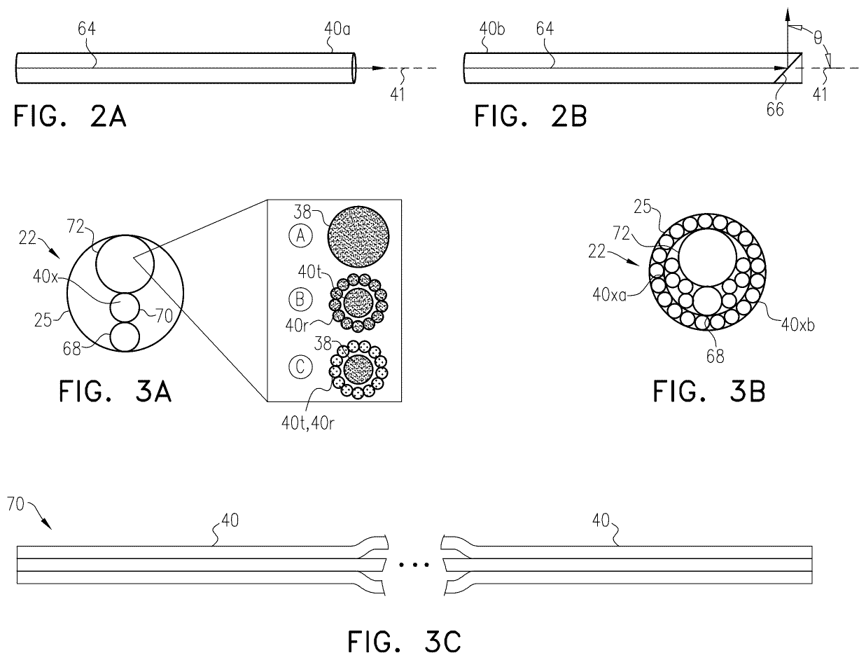

Reference is now made to FIGS. 2A and 2B, which schematically illustrate, respectively, two types of optical fibers 40a and 40b, in accordance with some embodiments of the present invention. Apparatus 21 (FIG. 1) comprises any one, or both, of these types.

In FIG. 2A, optical radiation 64 exits from optical fiber 40a in a direction parallel to the longitudinal axis 41 of the fiber at the distal end of the fiber. One or more optical fibers 40a may be used, for example, to probe and/or treat portions of the occlusion that are closer to the center of the blood vessel.

In contrast, in FIG. 2B, optical radiation 64 exits from optical fiber 40b at least partly laterally, i.e., at a nonzero angle θ with respect to longitudinal axis 41. For example, 0 may be between 1 and 135 degrees. In some embodiments, optical fiber 40b comprises a silica prism 66, an air pocket, or a polished distal end configured to provide optical radiation 64 with an at least partly lateral trajectory. One or more optical fibers 40b may be used, for example, to probe and/or treat portions of the occlusion that are closer to the periphery of the blood vessel.

Reference is now made to FIG. 3A, which schematically illustrates a frontal view of the distal end of catheter 22, in accordance with some embodiments of the present invention.

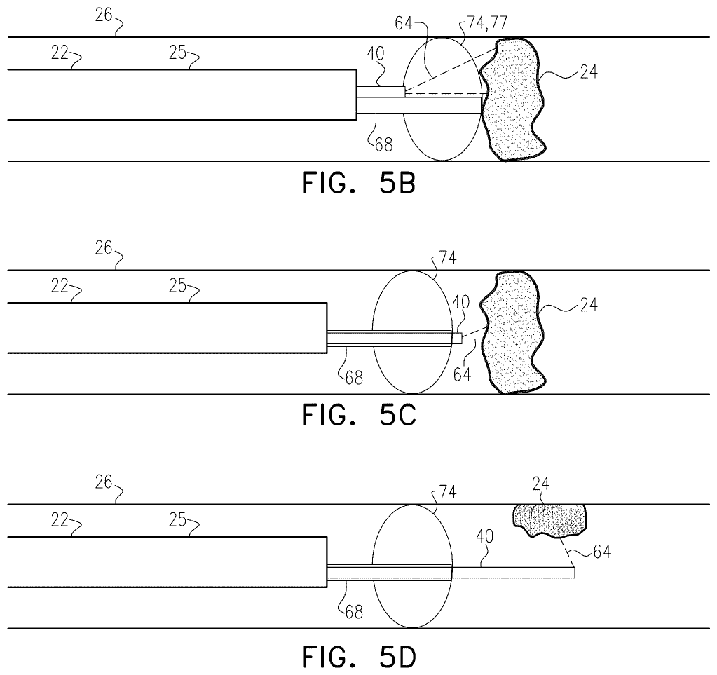

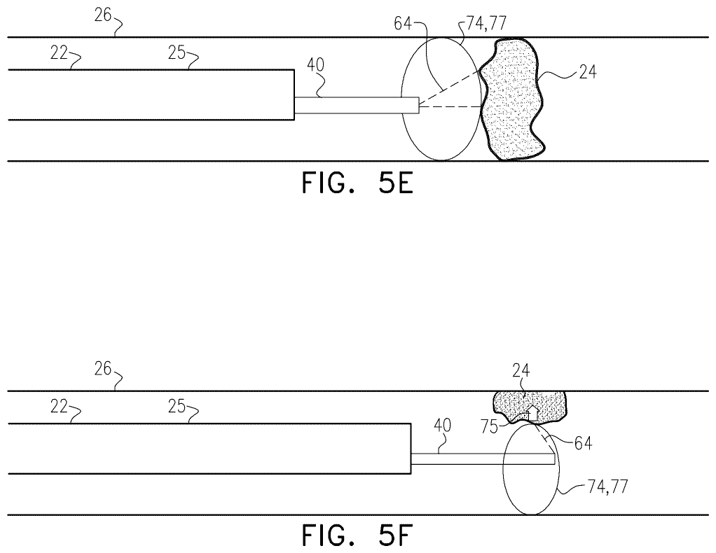

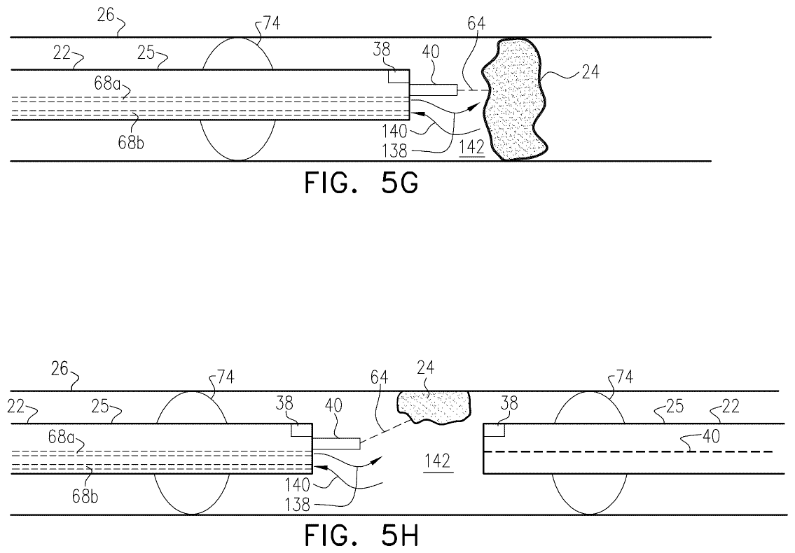

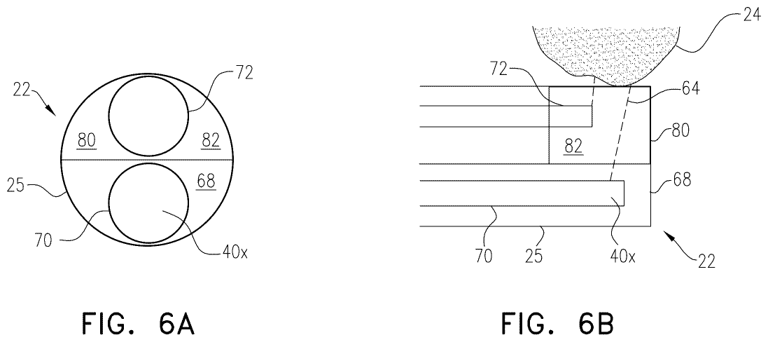

Catheter 22 comprises an outer tube 25. Typically, catheter 22 further comprises at least one working channel 68, which passes through outer tube 25. In some embodiments, working channel 68 comprises an inner tube that passes through, and is deployable from, outer tube 25. In other words, working channel 68 can be advanced from the distal end of the outer tube, e.g., as shown in FIGS. 5B-D, which are described below. In other embodiments, working channel 68 is a lumen of outer tube 25, i.e., the outer tube is shaped to define the working channel, e.g., as shown in FIGS. 6A-B, which are described below. In some embodiments, the diameter of the working channel is between 0.1 and 0.5 mm.

In some embodiments, a guidewire is passed through working channel 68, and the catheter is navigated over the guidewire. Alternatively or additionally, pump 30 (FIG. 1) applies suction through working channel 68 so as to pump blood proximally through the working channel. Alternatively or additionally, a liquid, such as saline, is delivered (e.g., pumped or infused) through the working channel. Alternatively or additionally, at least some optical fibers 40 pass through the working channel.

Typically, at least some of optical fibers 40 are arranged in one or more bundles 70, which run from the proximal end of the catheter to the distal end of the catheter. In some embodiments, as assumed in FIG. 3A, at least one bundle 70 comprises multiple treatment optical fibers 40x configured to direct treatment radiation at the occlusion. For example, in some embodiments, the treatment irradiation unit comprises multiple lasers, each being coupled to a different respective bundle 70.

In some embodiments, the diameter of each bundle 70 is between 0.1 and 2 mm. In some embodiments, at least one bundle 70 passes through working channel 68, as shown in FIGS. 6A-B, for example.

In some embodiments, for at least one bundle 70, the central portion of the bundle comprises one or more optical fibers 40a (FIG. 2A), while the more peripheral portion of the bundle comprises one or more optical fibers 40b (FIG. 2B). (To avoid confusion, it is noted that the modifiers “a” and “b” are used herein to differentiate between optical fibers with respect to the angle at which the optical radiation is directed, whereas the modifiers “x,” “t,” and “r” are used herein to differentiate between optical fibers with respect to their function.)

In some embodiments, for at least one bundle 70, one or more optical fibers in the bundle are configured to carry optical radiation independently from one or more other optical fibers in the bundle. For example, in some embodiments, shutters, which are disposed at the proximal ends of the optical fibers, are controlled such that optical radiation is emitted only into optical fibers 40a, only into optical fibers 40b, or into both types of optical fiber.

Typically, a probing module 72 runs from the proximal end of the catheter to the distal end of the catheter. In some embodiments, corresponding to option “A” in FIG. 3A, probing module 72 comprises camera 38, wiring 78 (FIG. 1), and, optionally, one or more optical fibers configured to direct illuminating light into the field of view of the camera. In other embodiments, corresponding to option “B” in FIG. 3A, probing module 72 comprises at least one transmission optical fiber 40t, which is configured to direct probing radiation at the occlusion, and surrounding receiving optical fibers 40r, which are configured to receive radiation that reflects, scatters, or fluoresces from the occlusion. In yet other embodiments, corresponding to option “C” in FIG. 3A, probing module 72 comprises camera 38 and surrounding optical fibers 40t and 40r. For both option B and option C, multiple transmission optical fibers 40t may be bundled together, and/or multiple receiving optical fibers 40r may be bundled together.

In some embodiments, one or more optical fibers are used for directing both probing radiation and treatment radiation at the occlusion.

Reference is now made to FIG. 3B, which schematically illustrates a frontal view of the distal end of catheter 22, in accordance with some embodiments of the present invention.

In some embodiments, rather than being bundled in bundle 70, optical fibers 40x, which direct treatment radiation, are distributed within the lumen of the catheter. For example, in some embodiments, optical fibers 40xb, which direct radiation at an angle as described above with reference to FIG. 2B, are distributed along the periphery of the lumen, while optical fibers 40xa, which direct radiation straight ahead as described above with reference to FIG. 2A, are distributed within a central portion of the lumen. (In such embodiments, catheter 22 does not necessarily comprise a physical partition between optical fibers 40xa and optical fibers 40xb.)

Reference is now made to FIG. 3C, which is a schematic illustration of a bundle 70 of optical fibers, in accordance with some embodiments of the present invention.

In some embodiments, at least two optical fibers 40 in bundle 70 are fused to one another at the proximal end of the catheter and/or at the distal end of the catheter, but not between the proximal end and the distal end. An advantage of the fusing at the proximal end and/or the distal end is that the optical radiation is carried more efficiently, given the proximity of the optical fiber cores. An advantage of the lack of fusion between the proximal end and the distal end is that the apparatus is more flexible, and thus, easier to navigate through the vasculature of the subject.

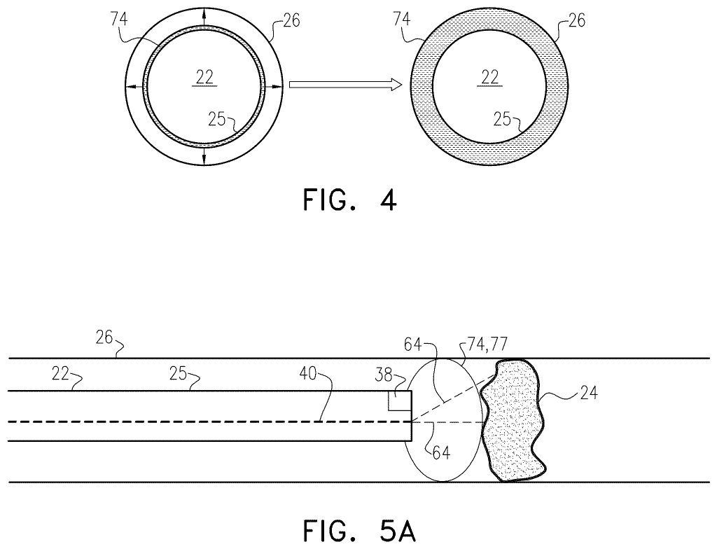

Reference is now made to FIG. 4, which is a schematic illustration of catheter 22 with a coupled expandable element 74, in accordance with some embodiments of the present invention.

In some embodiments, expandable element 74, comprising an inflatable element (e.g., a balloon) or a stent for example, is coupled to catheter 22. For example, expandable element 74 may be coupled to outer tube 25, e.g., within 1 mm of the distal tip of the outer tube. Alternatively, for embodiments in which working channel 68 comprises an inner tube, the expandable element may be coupled to the working channel. Alternatively, expandable element 74 is coupled to one or more optical fibers that pass through the catheter.

Expandable element 74 is expanded near the occlusion (e.g., within 1 cm, such as within 5 mm, of the occlusion), as shown at the left of FIG. 4, typically until the expandable element contacts the inner wall of blood vessel 26 over the entire circumference of the blood vessel, as shown at the right of FIG. 4. Typically, for embodiments in which expandable element 74 is coupled to the catheter, the expandable element stabilizes the catheter and/or centers the catheter within the blood vessel. Alternatively or additionally, expandable element 74 pushes blood away from, and/or blocks the flow of blood into, the area between the catheter and the occlusion, such that the contents of this work area can be modified more easily. For example, some or all of the blood in this work area may be replaced with a liquid (e.g., saline), as further described below with reference to FIG. 5G.

Alternatively or additionally, for some embodiments in which the expandable element comprises an inflatable element, the occlusion is irradiated via the inflatable element, and/or radiation returning from the occlusion is received via the inflatable element, as further described below with reference to FIGS. 5A-B and 5E-F. In general, the inflatable element is inflated with any fluid, such as air or saline, that facilitates the passage of optical radiation to and/or from the occlusion. (Typically, the catheter is shaped to define one or more fluid channels, and the fluid is pumped through the fluid channels.) Advantageously, the radiation-passage medium provided by the inflatable element may obviate the need to deliver saline or another liquid into the blood vessel.

Reference is now made to FIG. 5A, which is a schematic illustration of a use of expandable element 74, in accordance with some embodiments of the present invention.

In some embodiments, expandable element 74 comprises an inflatable element 77, which is coupled to the catheter (e.g., to outer tube 25) such that at least part of inflatable element 77 inflates distally to the catheter. Subsequently to the inflation of the inflatable element, optical radiation 64 is directed, via optical fibers 40, through the inflatable element and toward the occlusion. For example, the optical radiation may be directed through the inflatable element while the inflatable element contacts the occlusion.

In some embodiments, the optical radiation directed through inflatable element 77 includes probing radiation for probing the occlusion. Typically, in such embodiments, radiation that reflects, scatters, or fluoresces from the occlusion in response to the probing radiation is received, by optical fibers 40, via the inflatable element. Alternatively or additionally, the directed radiation includes treatment radiation for treating the occlusion.

Alternatively or additionally, camera 38 images the occlusion through the inflatable element, i.e., optical radiation returning from the occlusion is received, by the camera, via the inflatable element.

Reference is now made to FIG. 5B, which is a schematic illustration of another use of expandable element 74, in accordance with some embodiments of the present invention.

In some embodiments, expandable element 74, which comprises inflatable element 77, is coupled to working channel 68, rather than to outer tube 25 as in FIG. 5A. After the working channel is advanced from outer tube 25, the inflatable element is inflated, e.g., such that the inflatable element contacts the wall of blood vessel 26 and/or the occlusion. Subsequently, at least one optical fiber 40, which is advanced alongside the working channel or remains inside outer tube 25, directs radiation toward occlusion 24 through the inflatable element and, optionally, receives radiation from the occlusion through the inflatable element.

Reference is now made to FIGS. 5C-D, which are schematic illustrations of other uses of expandable element 74, in accordance with some embodiments of the present invention.

In some embodiments, after working channel 68 is advanced from outer tube 25, expandable element 74, which is coupled to the working channel, is expanded. Next, at least one optical fiber is advanced through the working channel and is then used to direct optical radiation toward the occlusion (and, optionally, receive radiation from the occlusion), as shown in FIG. 5C for a full occlusion and in FIG. 5D for a partial occlusion. Prior to directing the optical radiation at the occlusion, the blood in the work area is optionally diluted by a liquid (e.g., saline) that flows through the working channel.

Reference is now made to FIGS. 5E-F, are schematic illustrations of other uses of expandable element 74, in accordance with some embodiments of the present invention.

In some embodiments, expandable element 74, which comprises inflatable element 77, is coupled to the distal end of at least one optical fiber 40. After the optical fiber is advanced from outer tube 25, the inflatable element is inflated, e.g., such that the inflatable element contacts the wall of blood vessel 26 and/or occlusion 24. Subsequently, the optical fiber directs optical radiation toward occlusion 24 through the inflatable element and, optionally, receives radiation from the occlusion through the inflatable element, as shown in FIG. 5E for a full occlusion and in FIG. 5F for a partial occlusion.

Alternatively, the expandable element does not necessarily comprise an inflatable element. During the expansion of the expandable element, the expandable element uncouples from the optical fiber. The optical fiber can then be advanced or retracted through the expandable element prior to directing the optical radiation toward the occlusion.

As shown in each of FIGS. 5A-F, in some embodiments, the optical radiation is directed at least partly laterally toward the occlusion.

In some embodiments, expandable element 74 is used to push occlusion 24 toward the wall of the blood vessel, as indicated in FIG. 5F by a pushing indicator 75. Thus, advantageously, there may be no need to perform a separate angioplasty procedure. For example, after initially reducing the size of the occlusion (e.g., making a hole through the occlusion) by irradiation as described herein, the expandable element may be expanded within or adjacent to the remaining partial occlusion, such that the expandable element pushes the occlusion toward the periphery of the blood vessel.

Reference is now made to FIG. 5G, which is a schematic illustration of another use of expandable element 74, in accordance with some embodiments of the present invention.

In some embodiments, the expansion of expandable element 74, by virtue of pushing away blood and/or inhibiting the flow of blood, defines a work area 142 that includes the space between the expandable element and occlusion 24. Subsequently to the expansion of the expandable element, via catheter 22, a liquid (e.g., saline) is delivered (e.g., pumped or infused) into work area 142, as indicated by a first flow arrow 138, while blood (which is mixed with the liquid) is pumped from the work area, as indicated by a second flow arrow 140. For example, as shown in FIG. 5G, the liquid may be delivered through a first working channel 68a while the blood is pumped through a second working channel 68b. In this manner, the blood in work area 142 is diluted.

Subsequently to beginning the pumping of the blood, optical radiation 64 is directed, via at least one optical fiber 40, through work area 142 and toward the occlusion. In some embodiments, the optical radiation includes probing radiation for probing the occlusion. In such embodiments, typically, radiation that reflects, scatters, or fluoresces from the occlusion in response to the probing radiation is received through the work area and via at least one of the optical fibers. Alternatively or additionally, the optical radiation includes treatment radiation for treating the occlusion. In some embodiments, prior to directing the optical radiation, the optical fiber that is to direct the radiation is advanced from the catheter so as to facilitate the delivery of more radiation energy to the occlusion.

Alternatively or additionally, subsequently to beginning the pumping of the blood, camera 38 images the occlusion through the work area.

In some embodiments, the pumping is stopped once the concentration of the liquid in the work area is estimated to be greater than a predefined threshold. In other embodiments, the pumping of the blood (and delivery of the liquid) continues until the irradiation of the occlusion has finished.

In some embodiments, two expandable elements 74, which are coupled to different respective catheters 22, are expanded at opposite sides of a partial occlusion. In this regard, reference is now made to FIG. 5H, which is a schematic illustration of a use of two expandable elements, in accordance with some embodiments of the present invention.

In cases of a partial occlusion, it might be difficult to control the concentration of the liquid in work area 142 with only one expandable element. To address this challenge, in some embodiments, a second expandable element 74 is expanded at the opposite (downstream) side of the occlusion, such that the work area further includes the space between the second expandable element and the occlusion. Subsequently, while both expandable elements are expanded, optical radiation is emitted. Advantageously, the two expandable elements enclose the work area, such that the concentration of the liquid in the work area may be more easily controlled. Furthermore, the second expandable element inhibits any flow of debris from the work area.

In some embodiments, the second expandable element is coupled to a second catheter, which is inserted into blood vessel 26 from the same (upstream) or opposite (downstream) side of the occlusion. In some such embodiments, in addition to having a coupled expandable element, the second catheter is similar to the first catheter in other ways, e.g., by virtue of comprising at least one working channel, and/or by virtue of carrying a camera 38 and/or optical fibers 40. In some embodiments, while both expandable elements are expanded, optical radiation is directed at the occlusion from the first catheter, the second catheter, or both catheters, and/or the occlusion is imaged from the first catheter, the second catheter, or both catheters.

In other embodiments, a single catheter is used. For example, in some embodiments, the first and second expandable elements are coupled to the main body of a single catheter 22, the second expandable element being removably coupled to the catheter. The catheter is first advanced past the partial occlusion, to the downstream side of the occlusion, and the second expandable element is then expanded and uncoupled from the catheter. Subsequently, the catheter is retracted to the upstream side of the occlusion, and the first expandable element is then expanded. Alternatively, the second expandable element is coupled (removably or non-removably) to a working channel or optical fiber. The working channel or optical fiber is advanced to the downstream side, and the second expandable element is then expanded at the downstream side.

Reference is now made to FIG. 6A, which is a schematic illustration of a frontal view of the distal end of catheter 22, and to FIG. 6B, which is a schematic illustration of a side view of the distal end of catheter 22, in accordance with some embodiments of the present invention.

In some embodiments, the distal end of catheter 22 is shaped to define a chamber 80. In some such embodiments, chamber 80 contains a fluid 82, such as a liquid (e.g., saline) and/or air. Alternatively, chamber 80 is a vacuum chamber.

Optical fibers 40 pass, from one or more irradiation units, through the catheter to the distal end of the catheter. The optical fibers are configured to direct probing and/or treatment radiation emitted by the irradiation units through chamber 80 (e.g., through fluid 82) and toward occlusion 24. The distal end of each optical fiber can be either lateral or proximal to the chamber.

In some embodiments, the optical fibers are further configured to receive, through chamber 80, radiation that reflects, scatters, or fluoresces from the occlusion in response to the probing radiation, thereby facilitating probing the occlusion as described above with reference to FIG. 1. Alternatively or additionally, a camera, which belongs, for example, to probing module 72, is configured image the occlusion through the chamber.

For example, in some embodiments, the distal end of working channel 68 is lateral to chamber 80. A bundle 70 of optical fibers passes through working channel 68, such that chamber 80 is lateral to the distal ends of these optical fibers. In addition, the distal end of probing module 72, which comprises a camera and/or additional optical fibers as described above with reference to FIG. 3A, is disposed within chamber 80.

Typically, catheter 22 is advanced through the blood vessel until chamber 80 is adjacent to occlusion 24, e.g., until the wall of chamber 80 contacts the occlusion. Subsequently, the camera acquires images of the occlusion, and/or optical radiation is directed toward the occlusion from the optical fibers. Advantageously, chamber 80 provides a controlled medium for the passage of radiation to and from the occlusion.

In some embodiments, expandable element 74 surrounds catheter 22, as described above with reference to FIG. 4.

Reference is now made to FIG. 7, which is a schematic illustration of optical fibers 40 connected to console 43, in accordance with some embodiments of the present invention.

As described above with reference to FIG. 1, in some embodiments, treatment irradiation unit 42t comprises two lasers 84, such as a high-power pulsed laser and a mid-power continuous-wave laser. Alternatively or additionally, probing irradiation unit 42p comprises a broadband radiation source and/or an array 88 of low-power lasers. Typically, probing irradiation unit 42p is configured to emit optical radiation at multiple different wavelengths, pulse widths, powers, and/or numbers of pulses per second. Advantageously, this variation helps in deducing the composition of the occlusion.

Typically, connecting optical fibers 86 connect optical fibers 40 to console 43. Typically, lasers 84 are activated independently from each other, and are optically connected to different respective connecting optical fibers 86 (or to different respective bundles of connecting optical fibers). Probing irradiation unit 42p is optically connected to another optical fiber 86 (or to a bundle of connecting optical fibers), either directly or via one or more optical components 58.

For example, one connecting optical fiber 86 (or bundle of connecting optical fibers) may optically connect probing irradiation unit 42p to transmission optical fiber 40t and receiving optical fibers 40r (FIG. 3A). Additional connecting optical fibers (or bundles of connecting optical fibers) may connect treatment irradiation unit 42t to treatment optical fibers 40x (FIG. 3A).

Optical components 58 typically comprise one or more optical detectors 96, such as multiple optical detectors configured to detect different ranges of wavelengths. Typically, a small portion of the optical radiation emitted from the probing irradiation unit is deflected toward optical detectors 96, and optical detectors 96 measure the energy (or power) of this radiation. Alternatively or additionally, the optical detectors measure the energy (or power) of radiation returned from the occlusion. Alternatively or additionally, to facilitate monitoring for safety, optical detectors 96 measure the optical energy emitted by the treatment irradiation unit.

Typically, optical components 58 further comprise one or more spectrometers 92, such as multiple spectrometers configured to detect different ranges of wavelengths. The returning radiation is deflected toward spectrometers 92, and the spectrometers measure the energy of this radiation at multiple wavelengths.

For example, in some embodiments, most of the optical radiation emitted by probing irradiation unit 42p is polarized, while a small portion is unpolarized. Optical components 58 comprise a polarizing beamsplitter 90 comprising coating configured for each of the wavelengths emitted by the probing irradiation unit, or multiple polarizing beamsplitters for different ranges of the wavelengths. The polarized portion of the emitted radiation passes through polarizing beamsplitter 90 to the connecting optical fiber(s), typically via a lens 94, while the unpolarized portion is deflected, by the beamsplitter, to optical detectors 96. The returning radiation, which is not polarized, is deflected, by the beamsplitter, to spectrometers 92. (Alternatively to one or more beamsplitters, one or more coated mirrors can be used.)

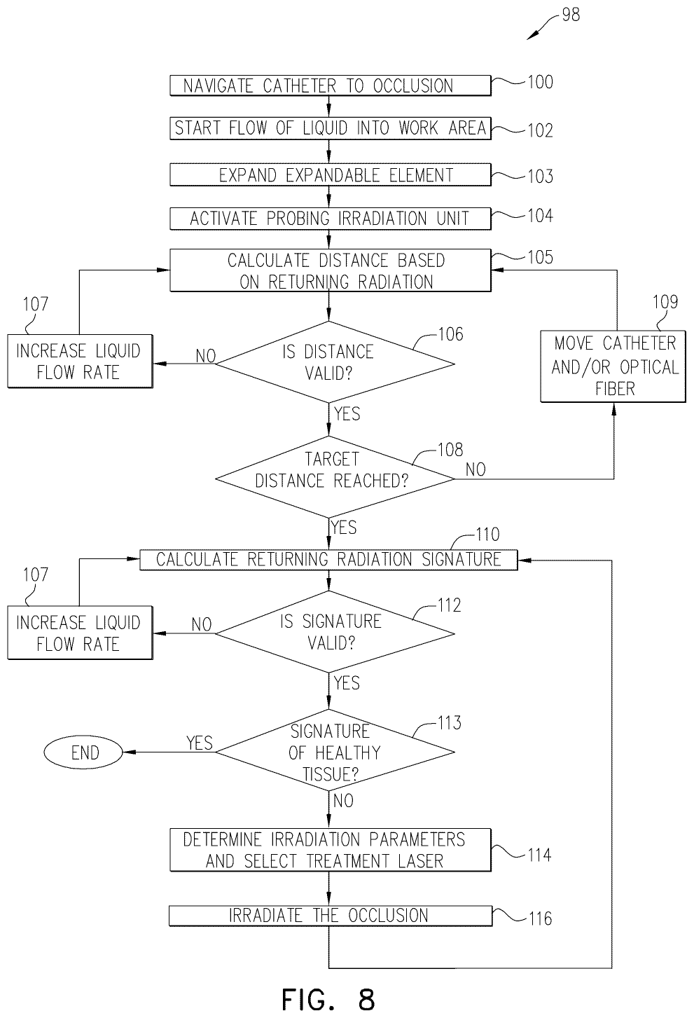

Processing unit 48 is connected to optical detectors 96 and to spectrometers 92. Typically, processing unit 48 computes the distance to the occlusion based on the output from optical detectors 96 and, optionally, spectrometers 92. For example, in some embodiments, the processing unit computes the distance based on the energy of the returning radiation at a particular wavelength, as measured by optical detectors 96 or spectrometers 92, normalized by the energy of the probing radiation, as measured by optical detectors 96. (Typically, the particular wavelength is one at which the amount of reflection, scattering or fluorescence is not dependent on the composition of the occlusion.) Alternatively or additionally, the processing unit computes a “signature” function, which includes the energy (or power) of the returning radiation at multiple wavelengths, as measured by the spectrometers, normalized by the energy of the probing radiation and by the distance to the occlusion. Based on the signature, the processing unit deduces the composition of the occlusion, as further described below with reference to FIG. 8.

In other embodiments, the probing radiation is modulated, and the calculation of the distance to the occlusion is based on the pulse modulation time travelled. In yet other embodiments, the distance calculation is based on the amount of returning radiation that reaches camera 38 (FIG. 1).

Reference is now made to FIG. 8, which shows a flow diagram for a method 98 for treating an occlusion, in accordance with some embodiments of the present invention.

First, at a navigating step 100, catheter 22 (FIG. 1) is navigated to the occlusion, typically over a guidewire. In some embodiments, this navigation is done under fluoroscopy, e.g., using radiopaque markers on the catheter. Alternatively or additionally, a location sensor, such as an electromagnetic location sensor, facilitates the navigation.

Next, in some embodiments, at a liquid-flowing step 102, the flow of a liquid (e.g., saline or contrast dye) into the work area (i.e., the area between the catheter and the occlusion), via working channel 68 (FIG. 3A), is begun. In some embodiments, the flow of the liquid continues until the end of the execution of method 98.

Next, at an expanding step 103, expandable element 74 (FIG. 4) is expanded. Alternatively, this step is performed before liquid-flowing step 102, or is omitted.

In some embodiments, a suction pump is activated before liquid-flowing step 102, between liquid-flowing step 102 and expanding step 103, or after expanding step 103. The suction of blood from the work area further expedites the dilution of the blood in this area.

Next, probing irradiation unit 42p (FIG. 7) is activated at an activating step 104, such that the probing irradiation unit begins emitting probing radiation. Typically, the probing irradiation unit remains active until the execution of method 98 ends. Alternatively (e.g., for embodiments in which the probing radiation and treatment radiation share the same optical fiber(s)), the probing irradiation unit is active only while treatment irradiation unit 42t (FIG. 7) is inactive, i.e., the two irradiation units alternate with one another.

In some embodiments, as assumed in FIG. 8, the probing radiation is directed at the occlusion by transmission optical fiber 40t, and the returning radiation is carried back to the proximal end of the catheter by receiving optical fibers 40r (FIG. 3A). In such embodiments, at a distance-calculating step 105, processing unit 48 (FIG. 7) calculates the distance from transmission optical fiber 40t to the occlusion based on the returning radiation. In some embodiments, the processing unit (e.g., cooperatively with processor 51) also displays this distance on display 62 (FIG. 1).

Next, at an evaluating step 106, the processing unit, or the user, evaluates whether the distance is valid. If not, the flow rate of the liquid (and/or the rate of suction pumping) is increased, at a flow-rate-increasing step 107, and distance-calculating step 105 is then repeated.

Once a valid distance is obtained, the processing unit checks, at a checking step 108, whether a target distance (or a range of target distances), which is typically predefined so as to optimize the treatment, was reached. If not, the catheter, and/or optical fiber 40t, is moved at a moving step 109, and distance-calculating step 105 is then repeated.

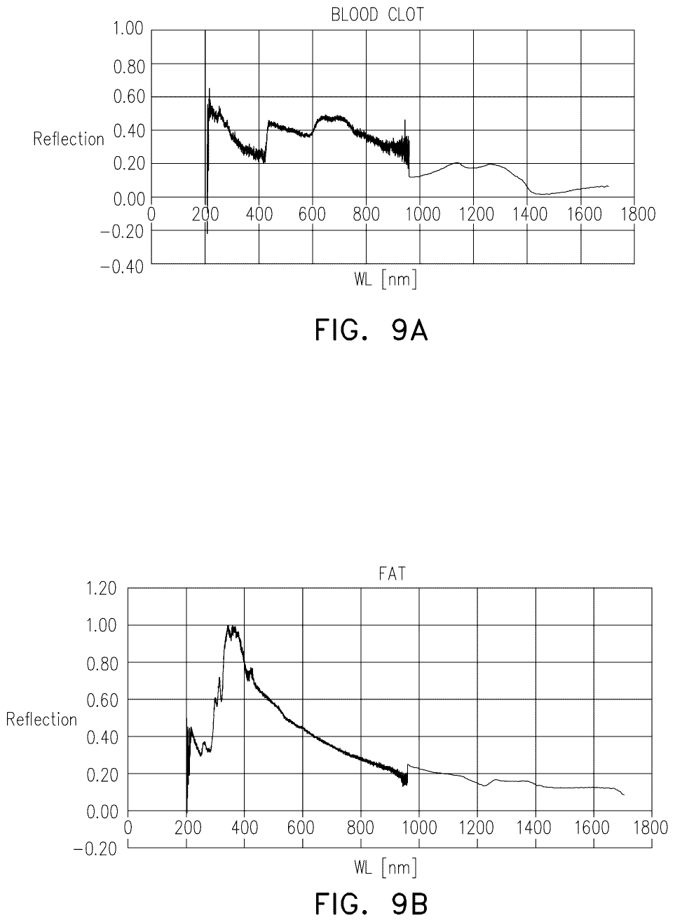

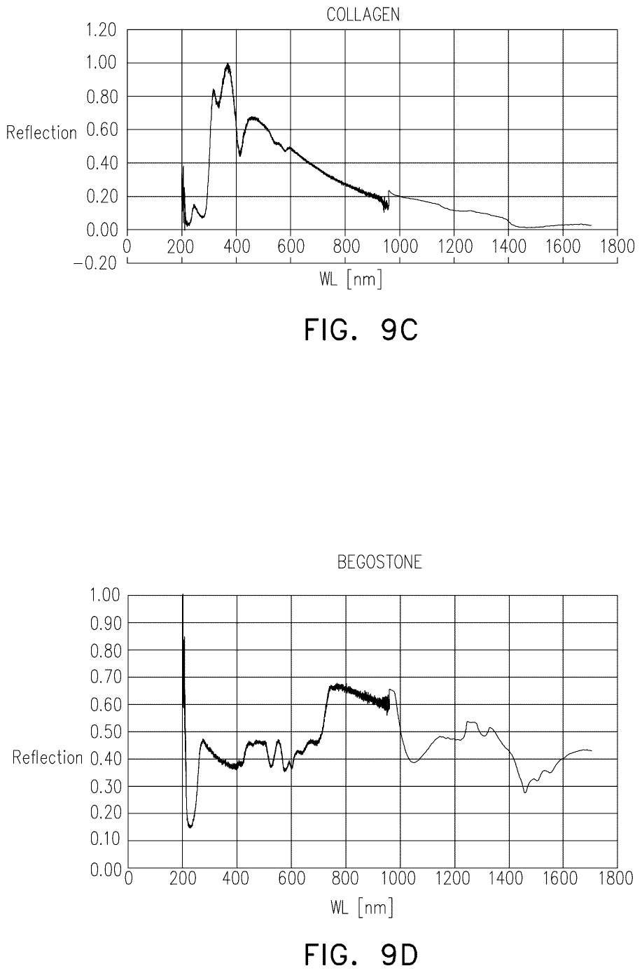

Once the target distance is reached, the processing unit calculates the signature of the returning radiation at a signature-calculating step 110. Subsequently, at an assessing step 112, the processing unit assesses whether the signature is valid, i.e., whether the signature corresponds to any one of multiple stored signatures for different respective materials, such as the signatures shown in FIGS. 9A-D. In some embodiments, this assessment is performed cooperatively with processor 51 (FIG. 1) and/or with a cloud-computing platform via Internet 52 (FIG. 1).

If the signature is not valid, flow-rate-increasing step 107 is performed, and signature-calculating step 110 is repeated. Otherwise, the processing unit checks, at a checking step 113, whether the signature is that of healthy tissue (e.g., blood or tissue of the blood-vessel wall), indicating that the occlusion was removed or that the catheter and/or optical fibers are not oriented properly. If yes, execution of method 98 ends. Otherwise, the composition of the occlusion (or at least the outermost layer of the occlusion) is ascertained to be that of the corresponding signature.

For example, if the calculated signature corresponds to the signature shown in FIG. 9A, the occlusion (or at least the outermost layer of the occlusion) is ascertained to be a blood clot.

In other embodiments, the composition of the occlusion is ascertained using other techniques, e.g., using imaging by camera 38 and/or by an extracorporeal imaging system.

Following the ascertaining of the composition, at a determining step 114, a set of one or more irradiation parameters that corresponds to the composition is determined. Typically, a treatment laser, which is configured to irradiate the occlusion in accordance with the irradiation parameters, is also selected. In some embodiments, the user is then asked to confirm the irradiation parameters.

Typically, the set of irradiation parameters is determined based on multiple predefined sets of one or more irradiation parameters, the sets corresponding to different respective materials and varying from each other with respect to the strength of the photoacoustic effect, relative to the strength of the photothermal effect, that the irradiation parameters provide. Typically, to determine the set of irradiation parameters, the processing unit either selects one of the predefined sets, or computes an average of one or more parameters over multiple ones of the predefined sets.

A photoacoustic effect is generated when a pulse of radiation forms a bubble in the medium (e.g., the blood, saline, or blood-saline mixture) near the occlusion, and the bubble then collapses, thereby generating a force that fragments the occlusion. In general, there is a tradeoff between the photoacoustic and photothermal effects, such that, for any given occlusion composition, some of the predefined sets of irradiation parameters provide a stronger photoacoustic effect but a weaker photothermal effect, while others provide a stronger photothermal effect but a weaker photoacoustic effect. For example, typically, a longer, lower-energy pulse provides a stronger photothermal effect, whereas a shorter, higher-energy pulse provides a stronger photoacoustic effect. In general, a stronger photoacoustic effect is preferred for harder materials, such as highly calcified (or collagenized) plaque. Hence, typically, the strength of the photoacoustic effect varies across the predefined sets such that any first predefined set, which corresponds to a harder material, provides a stronger photoacoustic effect, relative to any second predefined set corresponding to a softer material.

In some embodiments (e.g., for embodiments in which there is a range of target distances), the determination of the set of irradiation parameters is also in response to the distance from the occlusion.

In some embodiments, the irradiation parameters include a wavelength of the treatment radiation, the value of which may be between 270 and 3150 nm, for example. Alternatively or additionally, the irradiation parameters include a number of pulses per second, the value of which may be between 0.5 and 5000, for example. Alternatively or additionally, the irradiation parameters include a pulse energy, the value of which may be between 0.5 mJ and 10 J, for example. Alternatively or additionally, the irradiation parameters include a pulse width, the value of which may be between 1 us and 1 s, for example.

Next, at a treatment step 116, the occlusion is irradiated, via the optical fibers, in accordance with the determined set of irradiation parameters. In some embodiments, treatment step 116 includes advancing at least one optical fiber from the catheter, e.g., as shown in FIGS. 5B-H, prior to emitting the treatment radiation from the optical fiber. In such embodiments, the position of the distal end of the optical fiber is tracked, e.g., using control handle 23 (FIG. 1) or based on the reflection of probing radiation emitted by the optical fiber.

Following treatment step 116, the execution of method 98 returns to signature-calculating step 110, and, if necessary, another portion of the occlusion is then treated. In many cases, the composition of the occlusion is not uniform, such that different portions of the occlusion are treated with different treatment parameters.

Reference is now made to FIGS. 9A-D, which show experimental results illustrating the varying responses of different materials to irradiation, thereby demonstrating the utility of embodiments of the present invention.

Each of FIGS. 9A-D plots the amount of reflected probing radiation, which is normalized by the amount of transmitted probing radiation, as a function of the probing-radiation wavelength (WL). As can be seen, this function varies across different materials, such that the composition of the occlusion may be determined using multispectral probing as described herein. (With reference to FIG. 9D, it is noted that the chemical composition of BegoStone™ is similar to that of plaque.) Similar signature functions can be constructed for scattered and fluoresced radiation.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for treating an occlusion in a blood vessel, the apparatus comprising:
a catheter, configured for insertion into the blood vessel;
an irradiation unit;
one or more optical fibers, passing through the catheter, from the irradiation unit to a distal portion of the catheter; and
a processing unit configured to:
drive the irradiation unit to emit probing radiation such that the probing radiation is directed, by at least one of the optical fibers, toward the occlusion;
detect returning radiation which is returned via at least one of the optical fibers, in response to the probing radiation impacting the occlusion;
derive a composition of at least a portion of the occlusion based on a signature that is indicative of the composition within the returning radiation; and
drive the irradiation unit to irradiate the portion of the occlusion by emitting treatment radiation having a set of irradiation parameters via at least one of the optical fibers, the set of irradiation parameters being determined so as to modulate between a photoacoustic effect of the treatment radiation and a photothermal effect of the treatment radiation, based on the composition of the portion of the occlusion.

2. The apparatus according to claim 1, wherein the processing unit is configured to determine a distance from at least one of the optical fibers to the occlusion by analyzing the returning radiation.

3. The apparatus according to claim 1, wherein the processing unit is configured to derive a composition of at least a portion of the occlusion by comparing the signature within the returning radiation to multiple stored signatures for different respective materials.

4. The apparatus according to claim 1, wherein the processing unit is configured to drive the irradiation unit to irradiate respective portions of the occlusion with different treatment parameters, based on derived compositions of the respective portions.

5. The apparatus according to claim 1, wherein the irradiation unit comprises at least a high-power pulsed laser and a mid-power continuous-wave laser.

6. The apparatus according to claim 1, wherein the irradiation unit comprises at least a high-power pulsed laser, and wherein the processing unit is configured to irradiate the portion of the occlusion using the photoacoustic effect by driving radiation from the high-power pulsed laser, in response to deriving that the portion of the occlusion comprises calcified and/or collagenized plaque.

7. The apparatus according to claim 1, wherein the irradiation unit comprises at least a mid-power continuous-wave laser, and wherein the processing unit is configured to irradiate the portion of the occlusion using the photothermal effect by driving radiation from the mid-power continuous-wave laser, in response to deriving that the portion of the occlusion comprises soft tissue.

8. The apparatus according to claim 1, wherein the catheter is configured to facilitate creation of a work area, within which blood is removed or diluted, between tips of the one or more optical fibers and the occlusion.

9. The apparatus according to claim 8, wherein the apparatus comprises:
an expandable element coupled to the catheter or to one or more of the optical fibers and configured to expand within the blood vessel, thereby defining a space between the expandable element and the occlusion; and
at least one pump, configured to pump blood from the space, via the catheter, while a liquid flows into the space via the catheter, to thereby create the work area.

10. The apparatus according to claim 8, wherein the apparatus comprises an inflatable element coupled to the catheter or to one or more of the optical fibers, and configured to inflate within the blood vessel such that such that tips of the one or more optical fibers are disposed within the inflatable element, to thereby create the work area within the inflatable element.

11. The apparatus according to claim 8, wherein the distal portion of the catheter is shaped to define a chamber and the one or more optical fibers are configured to direct the optical radiation through the chamber and toward the occlusion, wherein the catheter is configured to facilitate creation of the work area by the chamber being placed adjacently to the occlusion.

12. A method for treating an occlusion in a blood vessel, the method comprising:
inserting a catheter into the blood vessel, with one or more optical fibers passing through the catheter, from an irradiation unit to a distal portion of the catheter;
driving the irradiation unit to emit probing radiation such that the probing radiation is directed, by at least one of the optical fibers, toward the occlusion;
detecting returning radiation which is returned via at least one of the optical fibers, in response to the probing radiation impacting the occlusion;
deriving a composition of at least a portion of the occlusion based on a signature that is indicative of the composition within the returning radiation; and
driving the irradiation unit to irradiate the portion of the occlusion by emitting treatment radiation having a set of irradiation parameters via at least one of the optical fibers; and
determining the set of irradiation parameters so as to modulate between a photoacoustic effect of the treatment radiation and a photothermal effect of the treatment radiation, based on the composition of the portion of the occlusion.

13. The method according to claim 12, further comprising determining a distance from at least one of the optical fibers to the occlusion by analyzing the returning radiation.

14. The method according to claim 12, wherein deriving the composition of at least the portion of the occlusion comprises comparing the signature that is indicative of the composition within the returning radiation to multiple stored signatures for different respective materials.

15. The method according to claim 12, wherein driving the irradiation unit to irradiate the portion of the occlusion comprises driving the irradiation unit to irradiate respective portions of the occlusion with different treatment parameters, based on derived compositions of the respective portions.

16. The method according to claim 12, wherein determining the set of irradiation parameters comprises determining that the set of irradiation parameters should include radiation from a high-power pulsed laser, in response to deriving that the portion of the occlusion comprises calcified and/or collagenized plaque.

17. The method according to claim 12, wherein determining the set of irradiation parameters comprises determining that the set of irradiation parameters should include radiation from a mid-power continuous-wave laser, in response to deriving that the portion of the occlusion comprises soft tissue.

18. The method according to claim 12, further comprising creating a work area, within which blood is removed or diluted, between tips of the one or more optical fibers and the occlusion.

19. The method according to claim 18, wherein creating the work area comprises:
expanding an expandable element coupled to the catheter or to one or more of the optical fibers and within the blood vessel, to thereby define a space between the expandable element and the occlusion; and
pumping blood from the space, via the catheter, while pumping a liquid into the space, to thereby create the work area.

20. The method according to claim 18, wherein creating the work area comprises inflating an inflatable element coupled to the catheter or to one or more of the optical fibers within the blood vessel such that such that the tips of the one or more optical fibers are disposed within the inflatable element, to thereby create the work area within the inflatable element.

21. The method according to claim 18, wherein the distal portion of the catheter is shaped to define a chamber and the one or more optical fibers are configured to direct the optical radiation through the chamber and toward the occlusion, and wherein creating the work area comprises placing the chamber adjacent to the occlusion.
